(19) United States
(12) Patent Application Publication
(10) Pub. No.: US 2019/0200954 A1
(43) Pub. Date: Jul. 4, 2019

(54) PROBE STRUCTURE

(71) Applicant: Neural Analytics, Inc., Los Angeles, CA (US)

(72) Inventors: Roman Flores, II, Los Angeles, CA (US); Michael Costa, Los Angeles, CA (US); Matthew Hutter, Los Angeles, CA (US); Kiah Lesher, Los Angeles, CA (US)

(21) Appl. No.: 16/236,013

(22) Filed: Dec. 28, 2018

Related U.S. Application Data
(60) Provisional application No. 62/612,029, filed on Dec. 29, 2017.

(51) Int. Cl. A61B 8/00; G01N 29/24; G01N 29/28; G01N 29/22; G01N 29/32; A61B 5/00

(52) U.S. Cl. CPC A61B 8/429; G01N 29/2487; G01N 29/28; A61B 2562/12; G01N 29/323; A61B 5/6843; A61B 8/4444; G01N 29/225

(57) ABSTRACT

The present disclosure relates to a probe structure that includes a probe configured to transmit or receive acoustic energy and having a first end and a second end opposite the first end, a probe hub defining a cavity for receiving at least a portion of the probe, and a joint coupled to the second end of the probe and configured to allow the probe to pivot within the probe hub.

### CROSS-REFERENCE TO RELATED PATENT APPLICATION

This application claims priority from U.S. provisional application No. 62/612,029, filed Dec. 29, 2017, which is incorporated herein by reference in its entirety.

### BACKGROUND

For devices utilizing a transducer or probe (e.g., optical devices, virtual reality headsets, surgical devices, ultrasound devices, imaging devices, automated Transcranial Doppler devices, and so on), there exist patient safety concerns and performance issues related to the placement and alignment of the probe against a subject (e.g., a subject's head). For example, the amount of pressure or force of the probe exerted against a subject can effect subject discomfort (e.g., due to excess force) and signal quality (e.g., due to insufficient force). However, some probe structures may not be capable of properly registering force against a subject due to off-axis torques or loadings applied at the probe surface, which can, in response, result in incorrect force compensation by way of too much force (causing patient discomfort) or too little force (causing poor probe performance).

### SUMMARY

In general, various embodiments relate to systems and methods for providing a probe structure capable of improved registration of off-axis torques or loadings at the probe surface. As such, by properly registering off-axis torque forces, appropriate compensation of the probe force can be accomplished.

According to some embodiments, a probe structure includes a probe configured to transmit or receive acoustic energy and having a first end and a second end opposite the first end, a probe hub defining a cavity for receiving at least a portion of the probe, and a joint coupled to the second end of the probe and configured to allow the probe to pivot within the probe hub.

In some embodiments, the joint includes a ball that is configured to allow the probe to pivot.

In some embodiments, the probe further includes an interface defining a recess that receives the ball of the joint.

In some embodiments, the interface includes a first piece and a second piece, the first piece defining a portion of the recess at a bottom hemisphere of the ball of the joint and the second piece defining a portion of the recess at a top hemisphere of the ball of the joint.

In some embodiments, the second piece of the interface partially envelops the top hemisphere of the ball of the joint such that the second piece restricts the ball within the recess.

In some embodiments, the first piece and the second piece are separate portions that are coupled together to form the interface.

In some embodiments, the ball of the joint is configured to rotate within the recess of the interface such that the probe rotates in a same direction as the ball of the joint does.

In some embodiments, the probe further includes a load cell coupled to the interface.

In some embodiments, at least the portion of the probe, the joint, the interface, and the load cell are axially aligned and housed in the cavity of the probe hub.

In some embodiments, the probe further includes a ring interposed between the second end of the probe and the interface.

In some embodiments, at least the portion of the probe, the joint, the interface, and the ring are housed in the cavity of the probe hub

In some embodiments, the cavity of the probe hub has a first inner diameter corresponding to a location of the ring within the probe hub and a second inner diameter corresponding to a location of at least the portion of the probe, and the first inner diameter is larger than the second inner diameter.

In some embodiments, the probe hub and at least the portion of the probe in the cavity of the probe hub define a gap between the probe hub and at least the portion of the probe to allow the probe to pivot within the probe hub.

In some embodiments, the gap is located around an entire circumference of the probe.

In some embodiments, the second end of the probe defines a hollow through which a protrusion of the joint is inserted.

In some embodiments, the protrusion and a ball are at opposite sides of the joint.

In some embodiments, the probe and the joint are coupled via the hollow and the protrusion.

In some embodiments, the protrusion and the hollow include corresponding threads such that the joint is configured to be screwed into the hollow.

In some embodiments, the probe is configured to transmit or receive ultrasound energy.

According to some embodiments, a method of manufacturing a probe structure includes providing a probe configured to transmit or receive acoustic energy and having a first end and a second end opposite the first end, providing a probe hub defining a cavity for receiving at least a portion of the probe, and coupling a joint to the second end of the probe, the joint configured to allow the probe to pivot within the probe hub.

### BRIEF DESCRIPTION OF THE FIGURES

Features and aspects will become apparent from the following description and the accompanying example embodiments shown in the drawings, which are briefly described below.

FIG. 1 illustrates a cross-sectional side view of a probe structure according to various embodiments.

FIG. 2 illustrates an enlarged cross-sectional side view of the probe structure shown in FIG. 1 according to various embodiments.

FIG. 3 illustrates a cross-sectional perspective view of the probe structure shown in FIG. 1 according to various embodiments.

FIG. 4 illustrates a side view of components of the probe structure shown in FIG. 1 according to various embodiments.

FIG. 5 illustrates a perspective view of the components of the probe structure shown in FIG. 4 according to various embodiments.

FIG. 6 to FIG. 16 illustrate various views of the probe structure shown in FIG. 1 according to various embodiments.

### DETAILED DESCRIPTION

The detailed description set forth below in connection with the appended drawings is intended as a description of various configurations and is not intended to represent the only configurations in which the concepts described herein may be practiced. The detailed description includes specific details for providing a thorough understanding of various concepts. However, it will be apparent to those skilled in the art that these concepts may be practiced without these specific details. In some instances, well-known structures and components are shown in block diagram form in order to avoid obscuring such concepts.

In the following description of various embodiments, reference is made to the accompanying drawings which form a part hereof and in which are shown, by way of illustration, specific embodiments that may be practiced. It is to be understood that other embodiments may be utilized, and structural changes may be made without departing from the scope of the various embodiments disclosed in the present disclosure.

In comparable probe structures, off-axis loads at a surface of a probe can create erroneous readings at a load cell coupled to the probe. For example, a load cell may register forces along an axis that is perpendicular to the surface of the probe (e.g., perpendicular along a z-axis through the load cell), and so off-axis force (e.g., force that is not normal to the surface of the probe) can therefore be registered erroneously at the load cell. For example, depending on the orientation of the off-axis load, the load cell can register the apparent force as greater than or less than the actual exerted force at the subject (e.g., at the subject's head). In some situations, the false force readings due to off-axis loads can cause the probe (e.g., via robotics) to become stuck in a loop as the probe is adjusted between applying too much force and too little force, rendering the probe dysfunctional.

In some embodiments, a probe structure is capable of properly registering off-axis torques or loads exerted on the surface of the probe (e.g., by a head of a patient). In some embodiments, the probe structure is configured to more accurately detect forces along an axis that is perpendicular to the surface of the probe by mitigating the erroneous effects of off-axis pressure at the surface of the probe. In some embodiments, the probe is continuously adjusted by robotics to maintain a normal position along a scanning surface and to maintain a suitable amount of pressure against the scanning surface, in response to force readings of the probe by the load cell.

According to various embodiments, the techniques and devices discussed herein can also be employed in various other embodiments using probes for medical and non-medical applications, such as, but not limited to, ultrasound, transcranial color-coded sonography (TCCS), phased arrays, and other known ultrasound energy modalities. Additionally, other techniques that use probes that emit or receive energy, such as, but not limited to, Near-Infrared Spectroscopy (NIRS), infrared, electrophysiological (EEG) monitoring, and so on can also be employed.

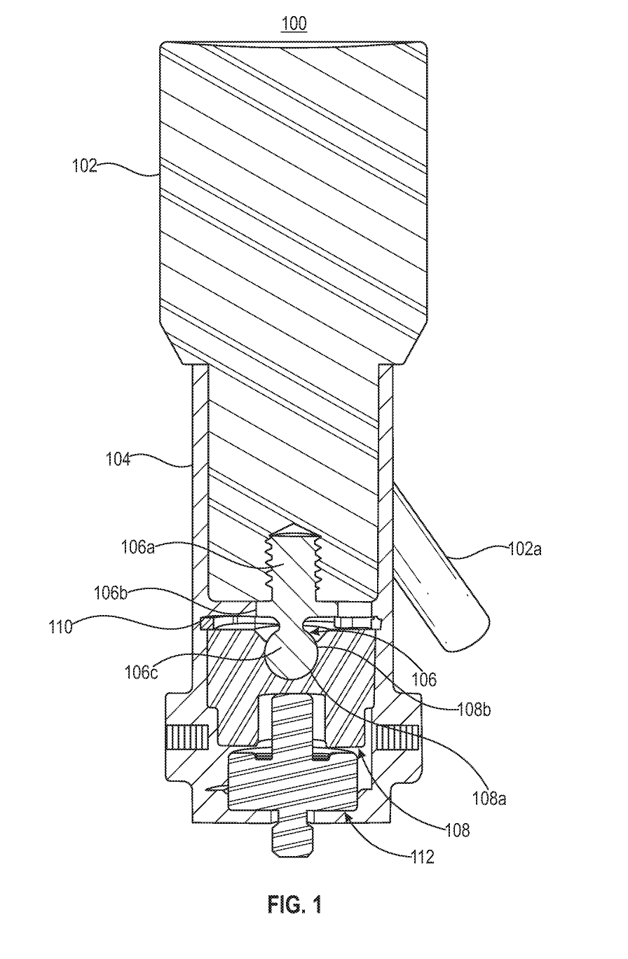

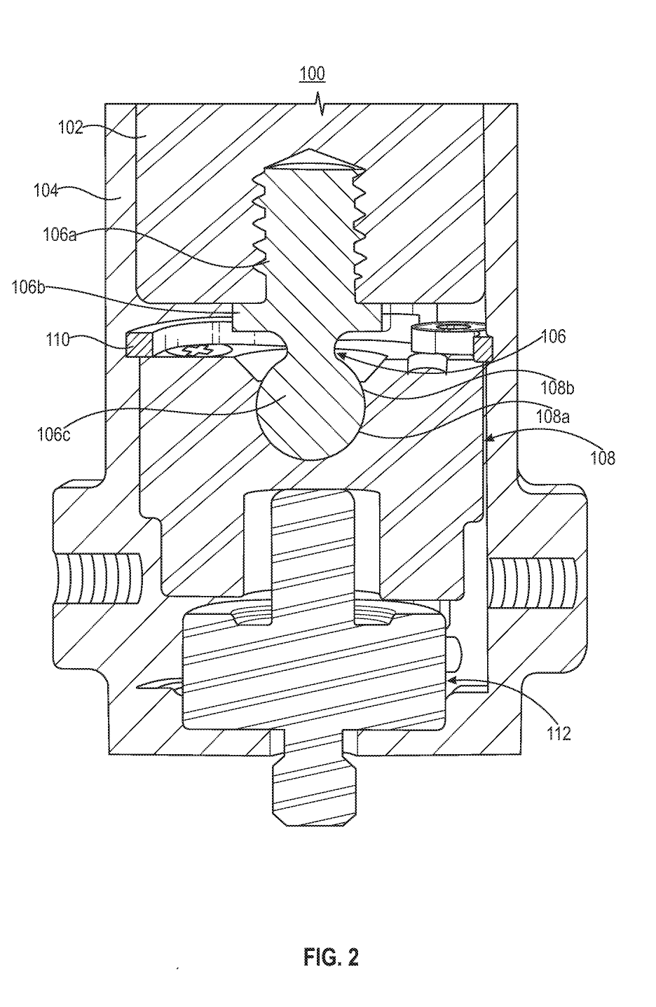

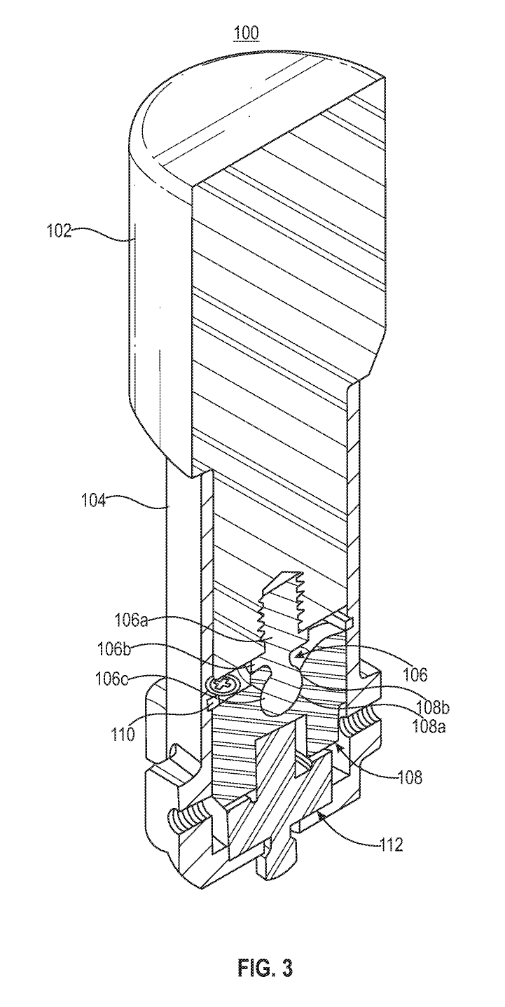

FIG. 1 illustrates a cross-sectional side view of a probe structure 100 according to various embodiments. FIG. 2 illustrates an enlarged cross-sectional side view of the probe structure 100 shown in FIG. 1 according to various embodiments. FIG. 3 illustrates a cross-sectional perspective view of the probe structure 100 shown in FIG. 1 according to various embodiments. In some embodiments, the probe structure 100 includes a probe 102, a probe hub 104, a joint 106, an interface 108, a ring 110, and a load cell 112.

In some embodiments, the probe 102 includes a first end (e.g., the end that is free and facing empty space) and a second end that is opposite to the first end. In some embodiments, the first end includes a concave surface that is configured to be adjacent to or contact a scanning surface (e.g., a subject's head). The concave surface is configured with a particular pitch to focus generated energy towards the scanning surface. In some embodiments, the probe structure 100 is a Transcranial Doppler (TCD) apparatus such that the first end of the probe 102 is configured to be adjacent to or contact and align along a human head (e.g., a side of the human head at a temporal acoustic window), and the first end of the probe 102 is configured to provide ultrasound wave emissions from the first end and directed into the human head (e.g., towards the brain). In other embodiments, the probe 102 is configured to emit other types of waves during operation, such as, but not limited to, infrared waves, x-rays, NIRS, electromagnetic, or the like. In other embodiments, the probe 102 includes a camera.

In some embodiments, the second end of the probe 102 is coupled to the joint 106. The probe 102 includes a hollow extending though the center of the probe 102. In some embodiments, the hollow 102 includes a threaded cavity-type interface. The hollow allows for alignment and fastening between the probe 102 and the joint 106. In other embodiments, the joint 106 is affixed to the probe 102 through an adhesive layer. The adhesive layer may be any suitable material for securely coupling the joint 106 and the probe 102 together, such as, but not limited to, an epoxy. In yet other embodiments, the probe 102 is secured to the joint 106 by any other suitable connecting means, such as, but not limited to, welding, potting, one or more hooks and latches, one or more separate screws, press fittings, or the like.

In some embodiments, the probe 102 (e.g., the TCD probe) has a tapered portion (e.g., the portion that becomes narrow when looking from the first end to the second end of the probe 102) that is configured to receive a cover. In some embodiments, the cover mounts snugly to the tapered portion to prevent a patient's skin from being pinched between the probe 102 and any other mechanism connected to the probe 102 (e.g., a robotic mechanism). Further, in operation, gel or other medium can be applied on the probe 102 and/or the patient's head to provide improved energy wave transmission between the head of the patient and the probe 102. Accordingly, in some embodiments, employing a cover snugly mounted at the tapered portion of the probe 102 prevents gel from moving past the tapered portion into the rest of the mechanism attached to the probe 102. For example, gel that travels beyond the tapered portion of the probe 102 may degrade operation of mechanisms (e.g., robotics) attached to the probe 102 or the probe structure 100 itself.

Beyond the tapered portion, the probe 102 (e.g., the TCD probe) extends into the probe hub 104. In some embodiments, the probe hub 104 is configured to mount with and allow for fastening of the probe hub 104 to a gimbal interface (e.g., of robotics). A data and/or power cable 102a extends from the probe 102 and through the probe hub 104 such that the cable 102a has proper clearance from the probe hub 104. In some embodiments, the data and/or power cable 102a allows for the flow of electricity to power the probe 102 and the flow of data from the probe 102 to corresponding electronics. In some embodiments, the cable 102a allows control signals to be provided to the probe 102.

In some embodiments, the probe hub 104 (e.g., gimbal) includes a pivoted support that allows for rotation of an object connected thereto (e.g., the probe 102), about one or more axes. For example, the probe hub 104 allows the probe 102 to pan, telescope, and/or tilt. Accordingly, in some embodiments, the probe hub 104 is coupled to robotics that move the probe 102 via the probe hub 104. Accordingly, in some embodiments, the probe hub 104 provides a plurality of single axis pivoted supports and interfaces with links and motors to allow pan, telescope, and/or tilt about respective X, Y, and/or Z axes. For example, the probe hub 104 further includes a gimbal interface for attaching to gimbal linkages that can control movement of the probe structure 100.

In some embodiments, the probe hub 104 has a fitted cavity for receiving and housing a portion of the probe 102, the joint 106, the interface 108, the ring 110, and the load cell 112, to provide security and alignment of the probe structure 100. The cavity of the probe hub 104 has a first inner diameter that corresponds to a location of the ring 110. The first inner diameter is substantially equal to (e.g., slightly larger than) an outer diameter of the ring 110 such that the ring 110 does not shift laterally or longitudinally while housed in the probe hub 104.

Similarly, the cavity of the probe hub 104 has a second inner diameter that corresponds to locations of the second end of the probe 102, the interface 108, and the load cell 112 when the probe 102, the interface 108, and the load cell 112 are housed within the probe hub 104. The second inner diameter is substantially equal to (e.g., slightly larger than) an outer diameter of the second end of the probe 102 and the interface 108. Accordingly, the probe 102, the joint 106, the interface 108, the ring 110, and the load cell 112 remain axially aligned within the probe hub 104. In some embodiments, the first inner diameter is greater than the second inner diameter.

In some embodiments, the probe hub 104 has a length long enough to encompass and house the load cell 112 (e.g., entirely), the interface 108 (e.g., entirely), the joint 106 (e.g., entirely), the ring 110 (e.g., entirely), and a portion (e.g., a substantial portion) of the probe 102. In some embodiments, the probe hub 104 is long enough to house approximately 50% of the length of the body of the probe 102. In other embodiments, the probe hub 104 is long enough to house more than 50% of the length of the body of the probe 102 (e.g., about 55%, 60%, 65%, or more). In other embodiments, the probe hub 104 houses less than 50% of the length of the body of the probe 102 (e.g., about 45%, 40%, 35%, or less). In particular embodiments, the probe hub 104 houses about 33% of the length of the body of the probe 102.

In some embodiments, the probe hub 104 includes a lengthwise slot. The slot may extend along the full length of the body of the probe hub 104. In other embodiments, the slot extends along less than the full length of the body of the probe hub 104. The slot is configured to receive and retain wires and cables originating from the components housed within the probe hub 104 (e.g., the cable 102a, wires from the load cell 112, and the like). Accordingly, the cables and wires of the probe structure 100 can be aligned and secured so that they do not become an obstacle during assembly or operation of the probe structure 100. In some embodiments, one or more of the wires or cables remains static in the slot, while one or more of the wires or cables is configured to move within the slot (e.g., flex or otherwise move along the length of the slot).

In some embodiments, the load cell 112 is located within the probe hub 104. In particular embodiments, the load cell 112 is fastened to the probe hub 104 (e.g., using adhesive, latches, screws, and the like). In some embodiments, the load cell 112 is a transducer that is used to translate physical phenomenon into an electrical signal that has a magnitude proportional to the force being measured. In some embodiments, wires extending from the load cell 112 provide electrical signals (e.g., data and power signals) emanating from the load cell 112 responsive to the force exerted on the load cell 112. In operation, when the probe 102 is pressed against a patient's head, a force will also be imparted through the joint 106 and the interface 108 to the load cell 112, resulting in a measurable electrical signal proportional to the force.

In some embodiments, a predetermined preload is applied to the load cell 112. For example, the load cell 112 may be designed to exhibit and include a preload in a range from about 2 Newtons to about 3 Newtons. In some embodiments, because the load cell 112 is aligned with and proximate the probe 102 (e.g., indirectly coupled to the probe 102), a force exerted against the concave surface of the first end of the probe 102, is registered and measured at the load cell 112.

Accordingly, in some embodiments, the probe structure 100 utilizes the measurements of the load cell 112 to adjust the pressure exerted by the probe 102 (e.g., via a robotic apparatus attached to the probe structure 100). For example, in some embodiments, the probe structure 100 decreases the force exerted against a human head by the probe 102 when the pressure measured by the load cell 112 is determined to be relatively high (e.g., the pressure measurement exceeds a predetermined threshold), or the probe structure 100 increases the force exerted against a human head by the probe 102 when the pressure measured by the load cell 112 is determined to be relatively low (e.g., the pressure measurement is below a predetermined threshold). In some embodiments, the predetermined threshold is user-defined and can be adjusted as desired.

In some embodiments, the load cell 112 includes a cylindrical protrusion extending upwards from the load cell 112. The protrusion passes into a recess of the interface 108 and extends therein. Accordingly, in some embodiments, the probe 102, the joint 106, the interface 108, and the load cell 112 remain aligned such that a maximum amount of perpendicular force is transferred from the surface of the probe 102 to the load cell 112. In some embodiments, the load cell 112 is affixed to a bottom inner surface of the probe hub 104 through an adhesive layer. The adhesive layer may be any suitable material for securely coupling the load cell 112 and the probe hub 104 together, such as, but not limited to, an epoxy, potting, and the like.

In some embodiments, the probe structure 100 is used in conjunction with robotics (e.g., the probe hub 104 is coupled to robotics). For example, the probe structure 100 is used in conjunction with a robotic arm (e.g., with multiple degrees of freedom, such as, but not limited to, six degrees of freedom). As another example, the probe structure 100 is used in conjunction with a robotic headset such as those described in non-provisional patent application Ser. No. 15/399,648, titled ROBOTIC SYSTEMS FOR CONTROL OF AN ULTRASONIC PROBE, filed on Jan. 5, 2017, and in non-provisional patent application Ser. No. 15/853,433, titled HEADSET SYSTEM, which are incorporated herein by reference in their entireties.

In between the second end of the probe 102 and the load cell 112 is an interface structure including the joint 106, the interface 108, and the ring 110. The joint 106 has a protrusion 106a, a nut 106b, and a ball 106c. In some embodiments, the protrusion 106a is configured to fit into the hollow of the second end of the probe 102. The protrusion 106a is threaded to allow the joint 106 to be secured to the probe 102 via corresponding threads in the hollow of the probe 102. Accordingly, the probe 102 and the joint 106 can be fastened together via the hollow of the probe 102 and the protrusion 106a of the joint 106. In other embodiments, the joint 106 and the probe 102 are fastened together by any other suitable method, such as, but not limited to, adhesive, welding, mechanical devices, and so on.

In some embodiments, the nut 106b allows for tightening of the joint 106 against the probe 102. For example, the nut 106b is a hex nut that allows a user to tighten the coupling strength between the probe 102 and the joint 106 using a tool (e.g., a wrench). In some embodiments, the ball 106c of the joint 106 has a substantially spherical shape and is attached to the nut 106b. In further embodiments, the ball 106c is configured to fit within a recess (e.g., a first recess) of the interface 108 so that the ball 106c can rotate in numerous axes while retained in the first recess of the interface 108. Accordingly, in some embodiments, the hex nut 106b is interposed between the protrusion 106a and the ball 106c. In some embodiments, the joint 106 is made from any suitable rigid material, such as, but not limited to, a metal, an alloy, and so on.

In some embodiments, the interface 108 has the first recess that is configured to receive and retain the ball 106c. In some embodiments, the first recess is shaped substantially similarly to the ball 106c, and an inner diameter of the first recess is slightly larger than the outer diameter of the ball 106c to allow the ball 106c freedom of movement within the first recess. In some embodiments, the interface 108 includes a first piece 108a and a second piece 108b. The first piece 108a defines a part of the first recess substantially corresponding to a bottom hemisphere of the ball 106c, and the second piece 108b defines a part of the recess substantially corresponding to a portion of the top hemisphere of the ball 106c directly above the bottom hemisphere of the ball 106c. Accordingly, in some embodiments, the second piece 108b of the interface 108 partially envelops the top hemisphere of the ball 106c (e.g., by forming an undercut portion therearound) and therefore captures and retains the ball 106c within the first recess, and restricts the ball 106c from moving upward from the interface 108.

In some embodiments, during manufacturing, the first piece 108a and second piece 108b are made separately and attached to each other thereafter. In some embodiments, the first piece 108a and the second piece 108b define one or more holes therethrough and the two pieces are attached to each other by one or more screws or bolts penetrating the one or more holes. In other embodiments, the first piece 108a and the second piece 108b are attached to each other by any other suitable method, such as, but not limited to, adhesive, welding, mechanical devices (e.g., latches), friction fitting, and the like.

In some embodiments, the interface 108 further defines a second recess opposite to the first recess (e.g., at a surface opposite to the surface of the interface 108 that defines the first recess). In some embodiments, a protrusion of the load cell 112 is configured to extend into the second recess of the interface 108. Accordingly, the ball 106c and the protrusion of the load cell 112 are proximate to each other with a section of the interface 108 interposed therebetween. In some embodiments, the interface 108 is made from any suitable material for promoting free rotational movement of the ball 106c within the first recess of the interface 108, such as, but not limited to, plastic (e.g., a slippery plastic, such as, polyoxymethylene, acetal, polyacetal, polyformaldehyde, and the like). In addition, in some embodiments, the material of the interface 108 has enough elasticity to allow the ball 106c to be pushed through the undercut portion of the first recess (e.g., by further separating the undercut portion) and such that the undercut portion returns to its original shape to retain the ball 106c within the first recess of the interface 108.

In some embodiments, the probe structure 100 defines a space or gap between the probe 102 and the probe hub 104 such that the probe 102 can move (e.g., minimally move) laterally between the inner surfaces of the probe hub 104. As such, because of the movement capability of the probe 102 within the probe structure 100 and the ball and socket structure provided by the joint 106 and the interface 108, the probe 102 can twist about a pivot point (e.g., the ball 106c) to mitigate off-axis downward pressure at the surface of the probe 102 so that the load cell 112 primarily or solely registers forces that are normal to the surface of the probe 102.

In some embodiments, the ring 110 has a C-shape. The ring 110 is configured and shaped to fit within the probe hub 104 at the portion of the probe hub 104 having the first inner diameter. Accordingly, the ring 110 serves as a locking mechanism that is configured to retain each of the components of the probe structure 100 in place within the probe hub 104. For example, because the ring 110 is slotted within the first inner diameter of the probe hub 104, and the remainder of the probe hub 104 has the second inner diameter that is more narrow than the first inner diameter, the ring 110 is held in place and therefore prevents the other components of the probe structure 100 from shifting upwards beyond the ring 110.

In some embodiments, the ring 110 contacts the interface 108 but does not contact the probe 102. In other embodiments, the ring 110 contacts the probe 102 and the interface 108. In other embodiments, the ring 110 does not contact the probe 102 or the interface 108. In some embodiments, the ring 110 has any suitable shape for securing the components of the probe structure 100 within the probe hub 104, such as, but not limited to, a circular hollow shape, a disk shape, a rectangular shape, and the like. In some embodiments, the ring 110 is made from any suitable rigid material for securing the components of the probe structure 100 within the probe hub 104, such as, but not limited to, plastic, metal, and the like.

As such, according to various embodiments, the probe structure 100 provides increased accuracy in readings due to the decoupling of off-axis loads at the surface of the probe 102 by using the joint 106 and the interface 108 structure interposed between the probe 102 and the load cell 112. In addition, in some embodiments, the probe structure 100 allows an operator to easily replace the probe 102 should it malfunction or become damaged, as the probe structure 100 does not require any use of adhesive between any of the components within the probe hub 104 (e.g., due to the use of the ring 110), and an operator can easily remove the probe 102 from the probe hub 104 and remove the joint 106 from the second end of the probe 102 and affix another working probe to the joint 106 for reinsertion into the probe hub 104.

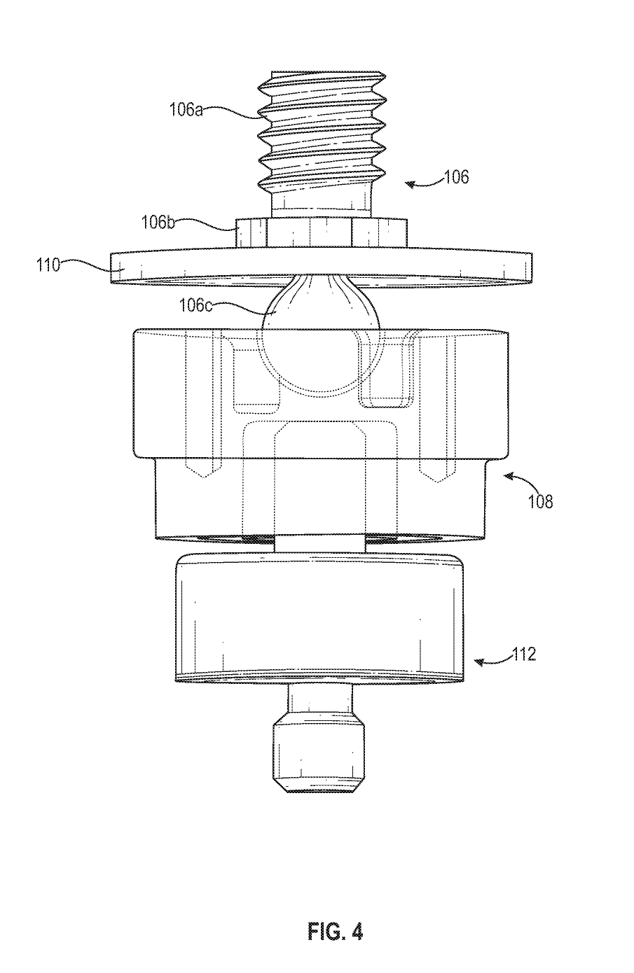

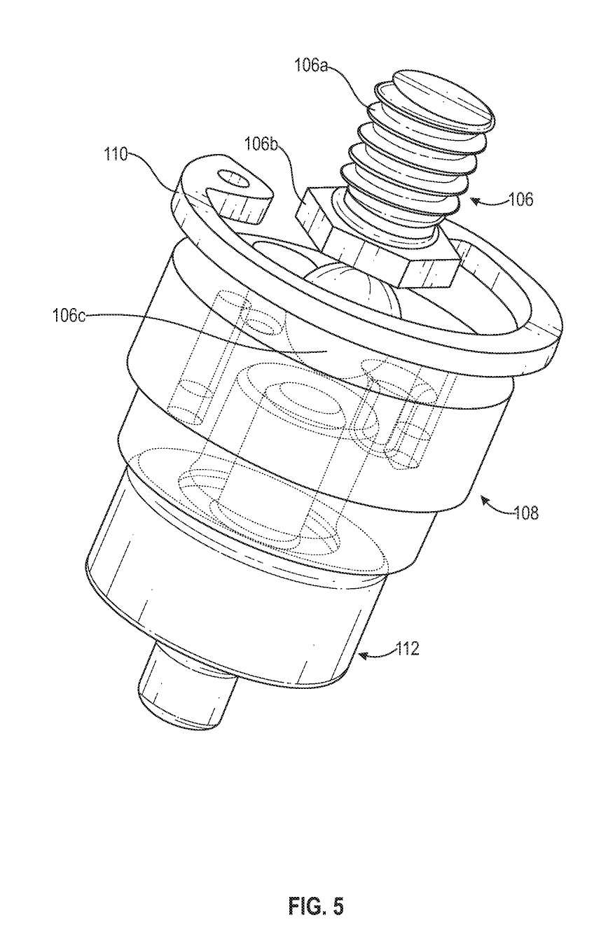

FIG. 4 illustrates a side view of components of the probe structure 100 shown in FIG. 1 according to various embodiments. FIG. 5 illustrates a perspective view of the components of the probe structure 100 shown in FIG. 4 according to various embodiments.

Referring to FIGS. 4 and 5, in some embodiments, illustrated are the components of the probe structure 100 including the joint 106, the interface 108, the ring 110, and the load cell 112. The interface 108 is transparent to reveal the first and second recesses thereof, which are configured to receive the ball 106c and the protrusion of the load cell 112, respectively.

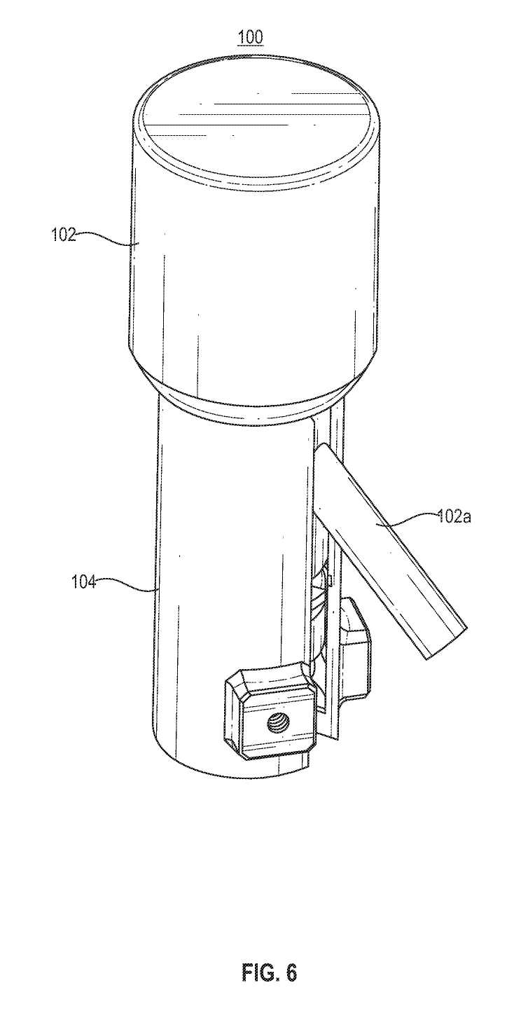

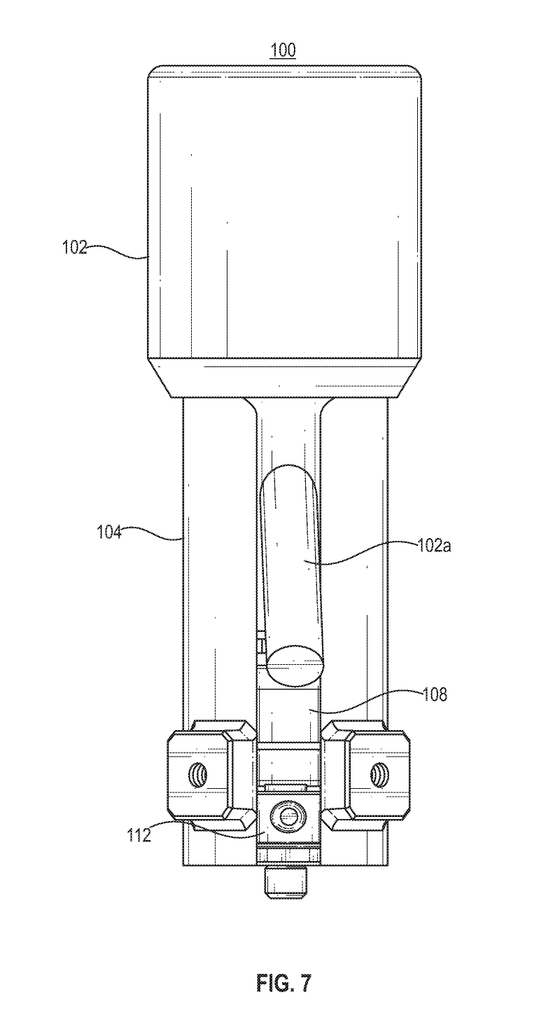

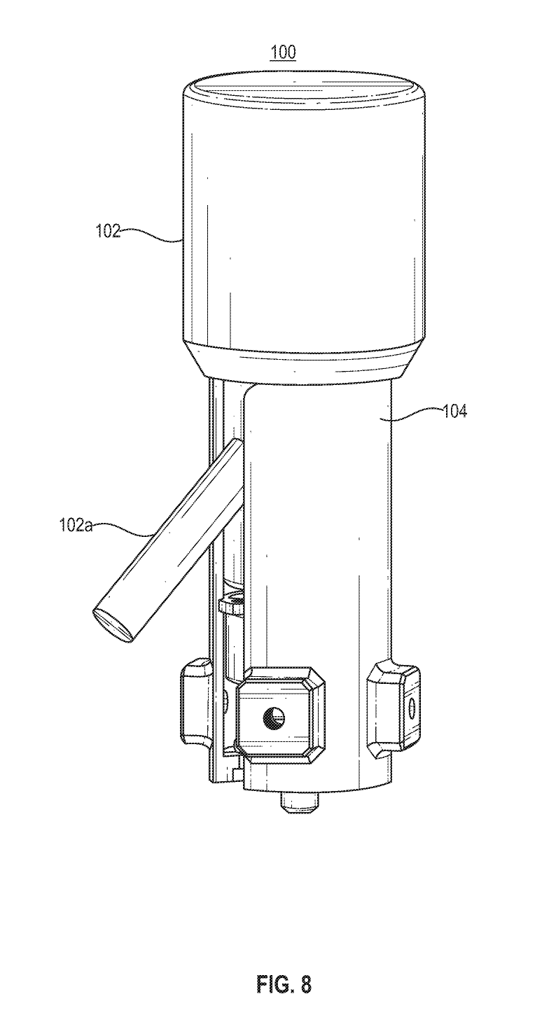

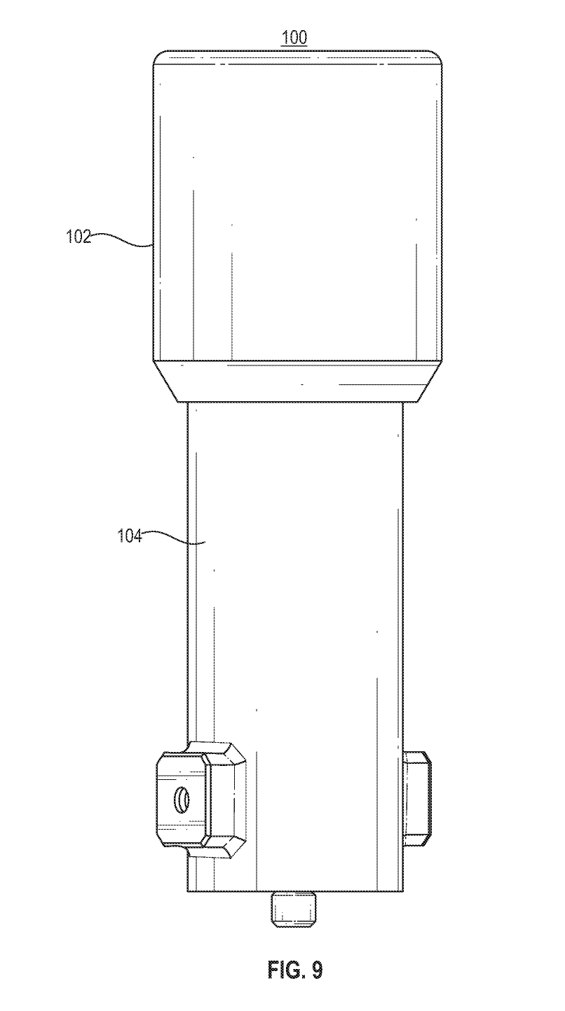

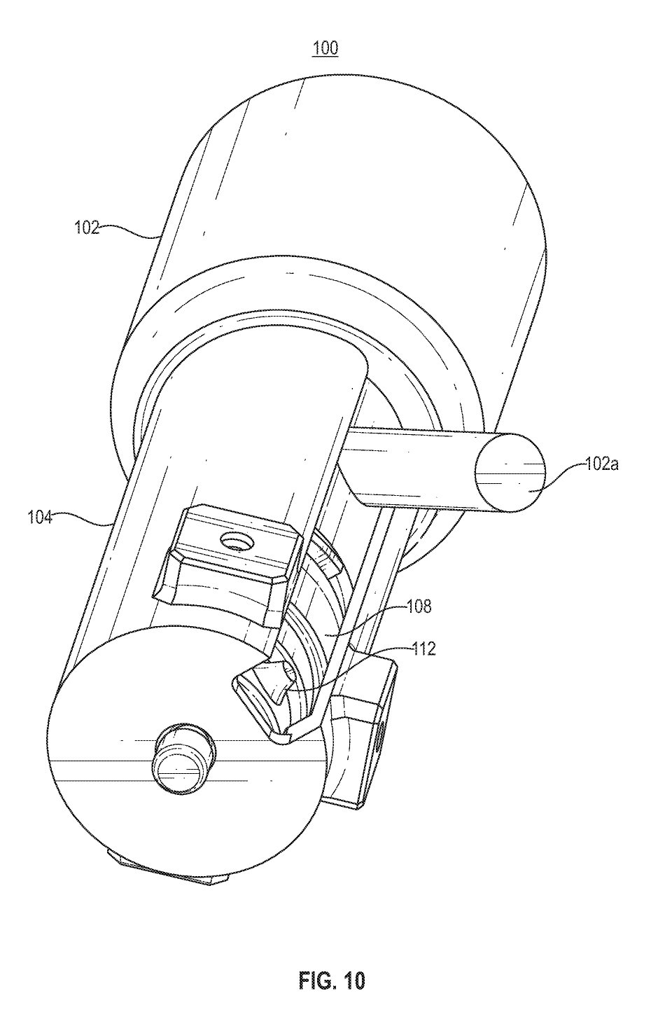

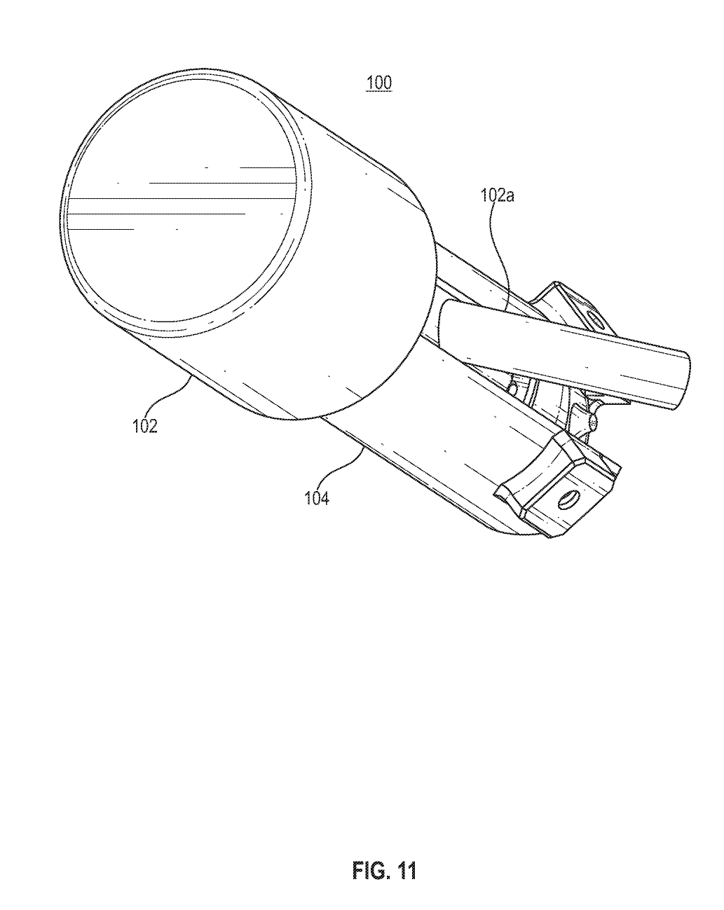

FIG. 6 to FIG. 16 illustrate various views of the probe structure 100 shown in FIG. 1 according to various embodiments.

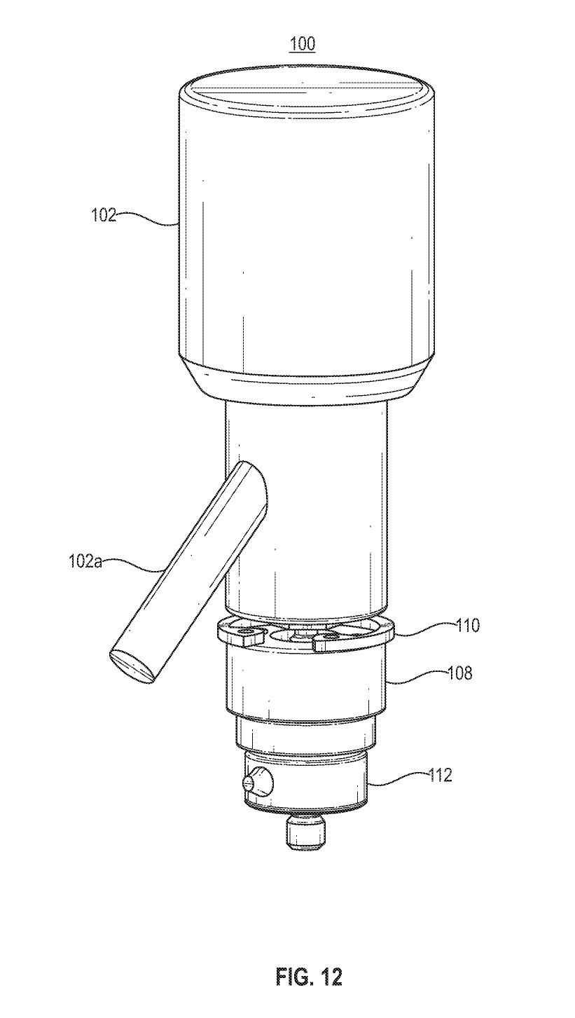

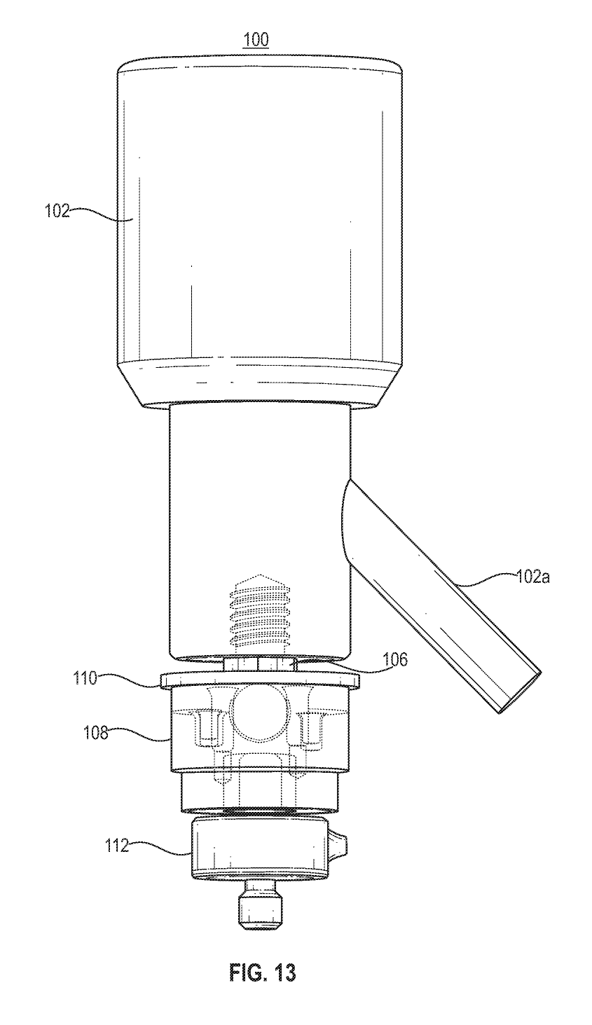

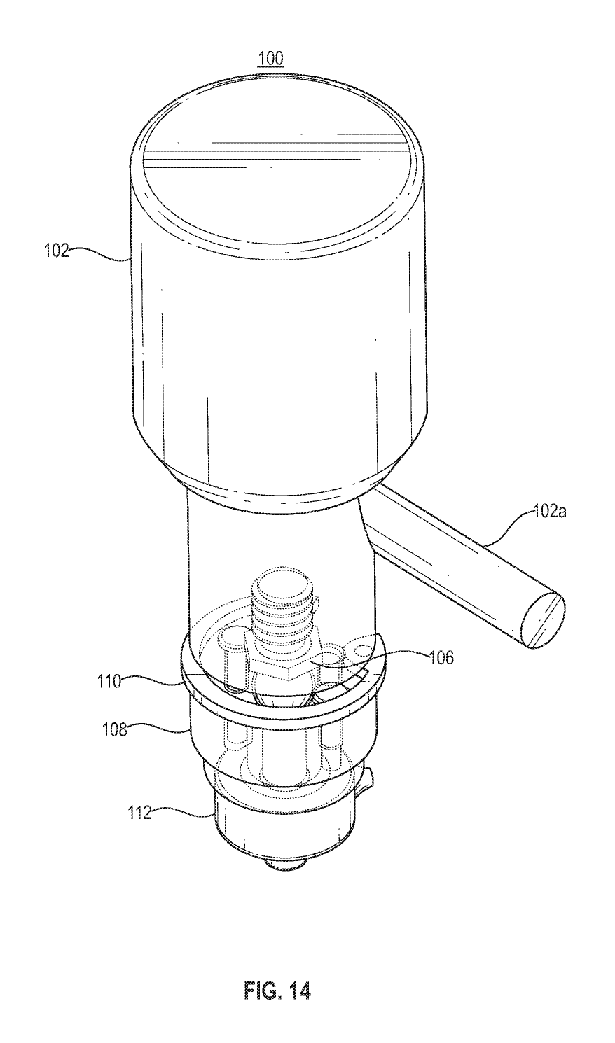

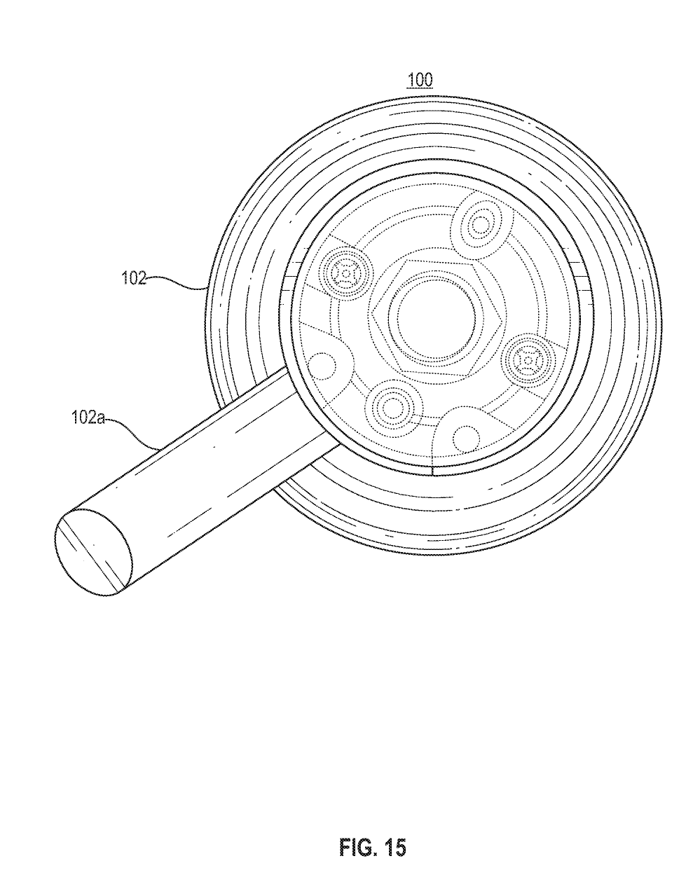

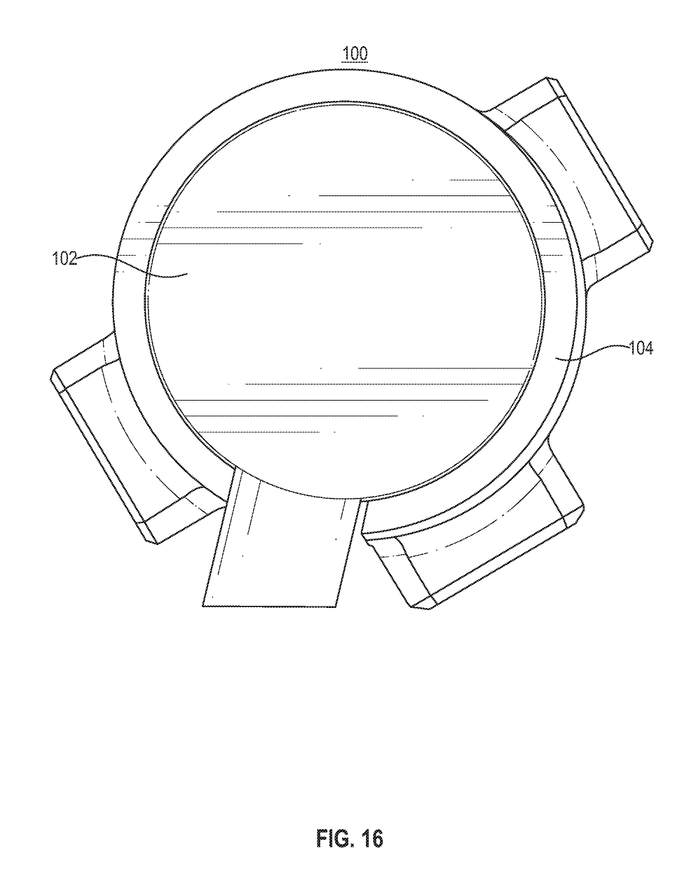

Referring to FIGS. 6-11, various external views of the complete probe structure 100, including the probe hub 104, are shown. Referring to FIG. 12, the probe structure 100 is shown without the probe hub 104, exposing the narrow section of the probe 102, the interface 108, the ring 110, and the load cell 112. Referring to FIGS. 13 and 14, the probe structure 100 is shown without the probe hub 104, and the probe 102 and the interface 108 are depicted as transparent, exposing the joint 106 therein. Referring to FIG. 15, the probe structure 100 is shown from an overhead view of the probe 102, and the probe 102 is depicted as transparent. Referring to FIG. 16, the probe structure 100 is shown as a cross-sectional view of the probe 102 in the probe hub 104, and depicts the space or gap that exists between the probe 102 and the probe hub 104 when the probe 102 is housed therein. The space or gap is located around an entire circumference of the probe 102.

As used herein, the terms “approximately,” “substantially,” “substantial” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same or equal if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

The above used terms, including “attached,” “connected,” “secured,” and the like are used interchangeably. In addition, while certain embodiments have been described to include a first element as being “coupled” (or “attached,” “connected,” “fastened,” etc.) to a second element, the first element may be directly coupled to the second element or may be indirectly coupled to the second element via a third element.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout the previous description that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

It is understood that the specific order or hierarchy of steps in the processes disclosed is an example of illustrative approaches. Based upon design preferences, it is understood that the specific order or hierarchy of steps in the processes may be rearranged while remaining within the scope of the previous description. The accompanying method claims present elements of the various steps in a sample order, and are not meant to be limited to the specific order or hierarchy presented.

The previous description of the disclosed implementations is provided to enable any person skilled in the art to make or use the disclosed subject matter. Various modifications to these implementations will be readily apparent to those skilled in the art, and the generic principles defined herein may be applied to other implementations without departing from the spirit or scope of the previous description. Thus, the previous description is not intended to be limited to the implementations shown herein but is to be accorded the widest scope consistent with the principles and novel features disclosed herein.

What is claimed is:

1. A probe structure comprising:
a probe configured to transmit or receive acoustic energy and having a first end and a second end opposite the first end;
a probe hub defining a cavity for receiving at least a portion of the probe; and
a joint coupled to the second end of the probe and configured to allow the probe to pivot within the probe hub.

2. The probe structure of claim 1, wherein the joint includes a ball that is configured to allow the probe to pivot.

3. The probe structure of claim 2, further comprising an interface defining a recess that receives the ball of the joint.

4. The probe structure of claim 3, wherein the interface comprises a first piece and a second piece, the first piece defining a portion of the recess at a bottom hemisphere of the ball of the joint and the second piece defining a portion of the recess at a top hemisphere of the ball of the joint.

5. The probe structure of claim 4, wherein the second piece of the interface partially envelops the top hemisphere of the ball of the joint such that the second piece restricts the ball within the recess.

6. The probe structure of claim 4, wherein the first piece and the second piece are separate portions that are coupled together to form the interface.

7. The probe structure of claim 3, wherein the ball of the joint is configured to rotate within the recess of the interface such that the probe rotates in a same direction as the ball of the joint does.

8. The probe structure of claim 3, further comprising a load cell coupled to the interface.

9. The probe structure of claim 8, wherein at least the portion of the probe, the joint, the interface, and the load cell are axially aligned and housed in the cavity of the probe hub.

10. The probe structure of claim 3, further comprising a ring interposed between the second end of the probe and the interface.

11. The probe structure of claim 10, wherein at least the portion of the probe, the joint, the interface, and the ring are housed in the cavity of the probe hub

12. The probe structure of claim 11, wherein the cavity of the probe hub has a first inner diameter corresponding to a location of the ring within the probe hub and a second inner diameter corresponding to a location of at least the portion of the probe, and the first inner diameter is larger than the second inner diameter.

13. The probe structure of claim 1, wherein the probe hub and at least the portion of the probe in the cavity of the probe hub define a gap between the probe hub and at least the portion of the probe to allow the probe to pivot within the probe hub.

14. The probe structure of claim 13, wherein the gap is located around an entire circumference of the probe.

15. The probe structure of claim 1, wherein the second end of the probe defines a hollow through which a protrusion of the joint is inserted.

16. The probe structure of claim 15, wherein the protrusion and a ball of the joint are at opposite sides of the joint.

17. The probe structure of claim 15, wherein the probe and the joint are coupled via the hollow and the protrusion.

18. The probe structure of claim 15, wherein the protrusion and the hollow include corresponding threads such that the joint is configured to be screwed into the hollow.

19. The probe structure of claim 1, wherein the probe is configured to transmit or receive ultrasound energy.

20. A method of manufacturing a probe structure, the method comprising:
providing a probe configured to transmit or receive acoustic energy and having a first end and a second end opposite the first end;
providing a probe hub defining a cavity for receiving at least a portion of the probe; and
coupling a joint to the second end of the probe, the joint configured to allow the probe to pivot within the probe hub.
